(19) United States
(12) Patent Application Publication
(10) Pub. No.: US 2011/0207814 A1
(43) Pub. Date: Aug. 25, 2011

(54) Combination Therapy for Breast Cancer Treatment

(76) Inventor: Pranela Rameshwar, Maplewood, NJ (US)

(21) Appl. No.: 12/977,036

(22) Filed: Dec. 22, 2010

Related U.S. Application Data
(60) Provisional application No. 61/289,561, filed on Dec. 23, 2009.

(51) Int. Cl. A61K 31/282; A61K 31/33; A61P 35/00

(52) U.S. Cl. 514/492; 514/183

(57) ABSTRACT

The present invention provides a novel treatment which features immune therapy in the context of the functions of MSCs as a novel approach to breast cancer treatment. By identifying and elucidating the role of MSCs in the behavior of breast cancer cells at metastatic sites and also at the primary region, this invention provides novel approaches for therapeutic intervention. More particularly, in the presence of MSCs a CXCR4 antagonist transitioned BCCs into cycling cells and conferred susceptibility to a chemotherapeutic agent. The proliferation of BCCs depended on the release of IL-1α and IL-1β from MSCs, but only if the CXCR4 antagonist uncoupled BCCs from MSCs.

### RELATED U.S. APPLICATION DATA

This application claims the benefit of U.S. Provisional application No. 61/289,561, filed Dec. 23, 2009, which is incorporated herein by reference in its entirety.

### GOVERNMENT SUPPORT

The research disclosed in this application was funded in part by Department of Defense Grants W81XWH-0810561 and W81XWH-0610689. Accordingly, the US government has certain rights in the invention.

### FIELD OF THE INVENTION

The present invention relates to a method for the treatment and mitigation of the progression of cancer, more particularly to a method for using Interleukin-1 to limit the response of mesenchymal stem cells to breast cancer cells.

### BACKGROUND OF THE INVENTION

Breast cancer survivors can develop metastasis after more than ten years of remission. The bone marrow is an organ for which breast cancer shows preference. Interestingly, in many cases, the bone marrow has been attributed as the source of breast cancer cells during breast cancer resurgence. It has been unclear if a particular subset of breast cancer cells survives as dormant cells in the bone marrow, and if so, how they can be targeted. Metastasis of breast cancer can occur without history of a primary tumor. Avoiding tertiary metastasis requires a thorough understanding of how breast cancer cells are protected towards dormancy, such as protection by cells in bone marrow.

Breast cancer cells can adopt quiescence at regions close to the endosteum. Changes in cytokine production and gap junctional intercellular communications (GJIC) have been implicated in breast cancer cell quiescence. Mesenchymal stem cells (MSCs) can contribute to breast cancer dormancy by virtue of its ability to interact with breast cancer cells through CXCR4 and the ligand, stromal cell-derived factor 1α (CXCL12). In addition, MSCs can protect breast cancer cells from immune response, thereby providing the breast cancer cells with an advantage to attain dormancy. CXCR4 is a seven-transmembrane, G-protein coupled receptor that facilitates chemoattaction of breast cancer cells to organs with high CXCL12 levels.

Upon entering bone marrow, MSCs can be among the first cells to interact with breast cancer cells since they are at the interface, around the main blood vessel. Here the immune suppressive effects of MSCs can provide an immediate advantage by protecting the breast cancer cells from immune clearance. This will give the breast cancer cells an advantage to survive in bone marrow and integrate in regions close to the endosteum as dormant cells.

MSCs are important for blood vessel integrity, thereby making them unlikely drug discovery targets. Similarly, direct targeting of GJIC between breast cancer cells and stroma could be toxic since gap junctions between stromal cells are important for hematopoietic support. Thus, there is a current need to identify and understand the role of MSCs in the behavior of breast cancer cells at metastatic sites and also at the primary region in order to provide novel methods of therapeutic intervention.

### BRIEF SUMMARY OF THE INVENTION

The present invention relates to a method of treating breast cancer which comprises dissociating quiescent breast cancer cells from their environment in the bone marrow and treating the disassociated breast cancer cells with standard chemotherapeutic agents.

More particularly, the invention relates to a method which comprises using a CXCR4 antagonist, such as, for example AMD3100 to break a BCC-MSC association, thus dissociating the quiescent breast cancer cells and inducing breast cancer cell proliferation and susceptibility to chemotherapy.

Thus the present invention relates to a novel immune therapy for cancer using Interleukin-1 to limit the response of mesenchymal stem cells to breast cancer cells. It has now been shown that MSCs support the growth of breast cancer cells via the production of IL-1 and make the breast cancer cells susceptible to carboplatin treatment. Thus the present invention provides a novel treatment which features immune therapy in the context of the functions of MSCs as a novel approach to breast cancer treatment. By identifying and elucidating the role of MSCs in the behavior of breast cancer cells at metastatic sites and also at the primary region, this invention provides novel approaches for therapeutic intervention. More particularly, in the presence of MSCs a CXCR4 antagonist transitioned BCCs into cycling cells and conferred susceptibility to a chemotherapeutic agent. The proliferation of BCCs depended on the release of IL-1α and IL-1β from MSCs, but only if the CXCR4 antagonist uncoupled BCCs from MSCs.

### BRIEF DESCRIPTION OF THE DRAWINGS

FIG. 1 shows CXCL12-CXCR4 interaction between BCCs and MSCs in BCC proliferation.

FIG. 2 shows the effect of AMD3100 on BCC proliferation following extended co-culture with MSCs.

FIG. 3 shows the effect on cycling of BCCs by AMD3100.

FIG. 4 show IL-1 in AMD3100-mediated BCC cycling.

FIG. 5 shows the treatment of co-cultured BCCs-MSCs with carboplatin and AMD3100.

FIG. 6 shows the resistance to chemotherapy in non-combinatorial, AMD3100-treated MSC/BCC co-cultures.

FIG. 7 shows the effects of AMD3100 and carboplatin on tumor volume.

### DETAILED DESCRIPTION OF THE INVENTION

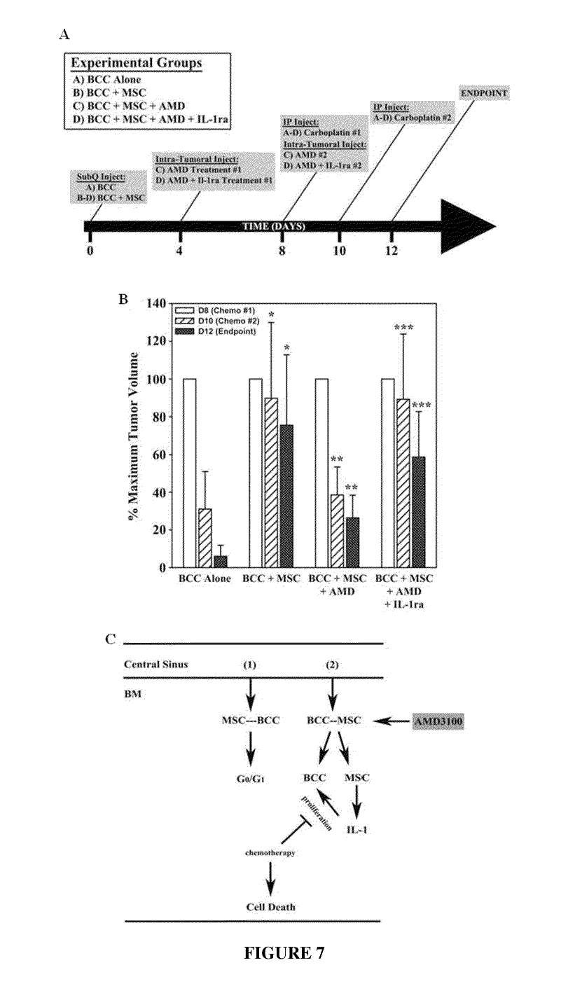

MSCs can pose a significant clinical dilemma for breast cancer treatment, due to MSCs acting as suppressor of breast cancer cells proliferation. In the absence of proliferation, the breast cancer cells will show a seemingly dormant phenotype and could be refractory to most anti-cancer agents. New therapies are required to reverse the dormant breast cancer cells into proliferation cells for targeting. In the studies which yielded the present invention, we showed CXCL12 and its receptor, CXCR4, as mediators in the interactions between MSCs and breast cancer cells, leading to growth arrest (FIG. 7C, part 1). Pharmacological disruption with AMD3100, (FIG. 7C, part 2) stimulated breast cancer cells proliferation through MSC-derived IL-1α and IL-1β. This renders the actively cycling breast cancer cells to be susceptible to carboplatin.

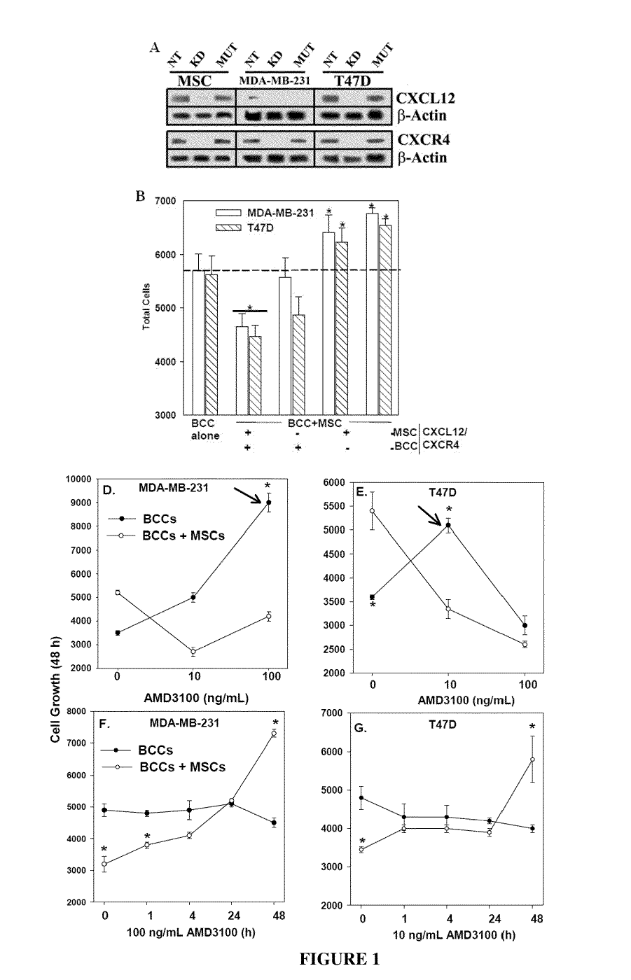

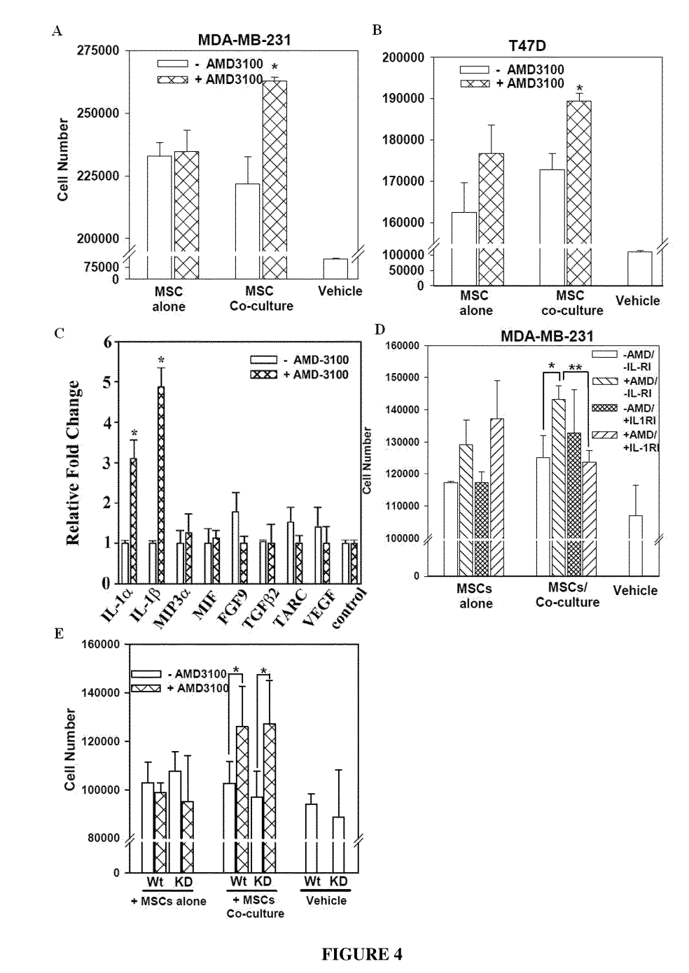

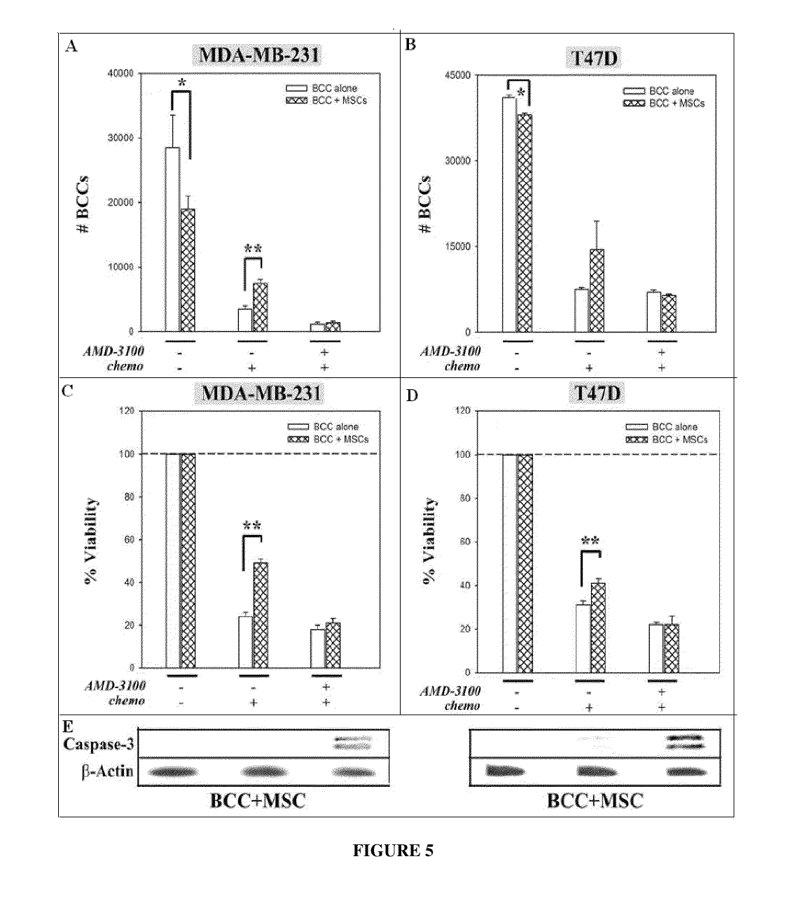

MDA-MB-231 seems to be more responsive to AMD3100 than T47D (FIG. 1). This might be due to single interaction between CXCL12 and CXCR4 between MSCs and MDA-MB-231 since this cell line is null for CXCL12. Cell cycle arrest of breast cancer cells depends on CXCL12-CXCR4 interaction; and re-entry into cycling by AMD3100 requires the presence of MSCs since the antagonist alone shows not change (FIG. 4B). Both the triple hormone receptor positive and negative breast cancer cells showed comparable outcome with AMD3100 and carboplatin (FIG. 5). The 12-day co-culture is significant because clinically, it is expected that breast cancer cells will be in contact with MSCs for prolonged periods; which is consistent with studies in which targeting of CXCR4 prevents metastasis of breast cancer to the lung.

Interestingly, AMD3100 alone reduced the growth of the breast cancer cells cultured alone (FIGS. 1D and 1E). We surmised that CXCR4 might be activated through autocrine and/or paracrine stimulation to activate cell growth, which would be blocked by AMD3100. In contrast, the antagonist ‘uncouples’ MSCs from breast cancer cells to ‘free’ the MSCs for support of breast cancer cells growth (FIG. 7C).

The studies are significant to breast cancer dormancy in any organ, but specifically for bone marrow. In addition to acting as an antagonist to CXCR4, AMD3100, also known as Plerixafor, is a macrocyclic compound which is an allosteric agonist of CXCR7. Our studies do not indicate that AMD3100 activates CXCR7 for the proliferation of breast cancer cells since alone, breast cancer cells show suppressed growth with AMD3100 (FIG. 1). The data favor MSC-mediated factors in the growth of breast cancer cells by AMD3100. Given this, it is not a surprise that AMD3100 can decrease primary and metastatic breast cancer in mice, but with no effect on the overall survival for experimental lung metastases. The studies add to the indication for AMD3100.

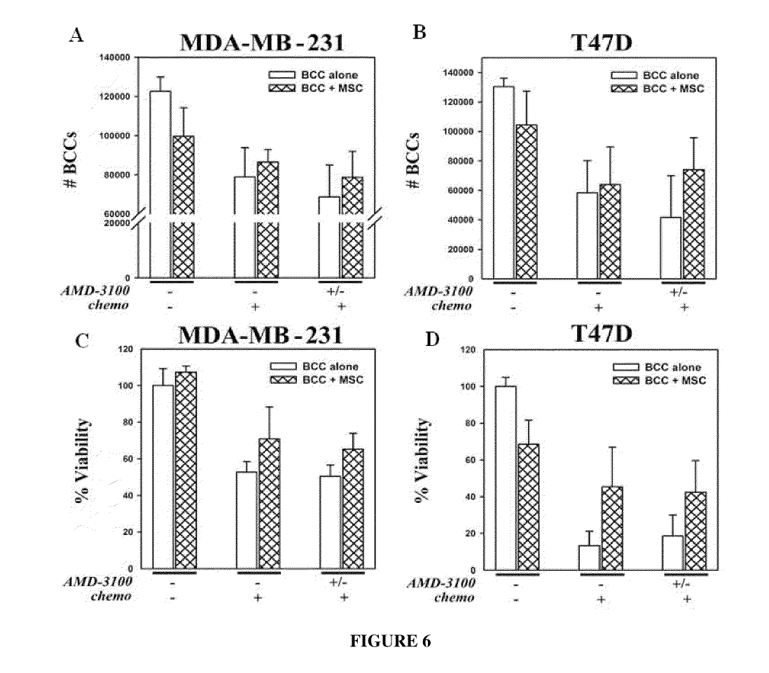

Indeed, an interesting and unexpected observation is the need for continued presence of AMD3100 during treatment with carboplatin. This suggests that the interaction between breast cancer cells and MSCs is rapid. Pre-treatment with the antagonist for 48 hours prior to chemotherapy did not induce significant cell death (FIG. 6) as compared to co-treatment (FIG. 5). The clinical significance of this result is that therapies targeting CXCR4 to promote re-cycling of dormant breast cancer cells may need to be delivered as an adjuvant to chemotherapy to achieve efficiency.

We determined that for the highly aggressive MDA-MD-231 breast cancer cells, treatment of co-cultures with AMD3100 promoted the subsequent release of IL-1α and IL-1β from the MSCs (FIG. 6D). This finding warrants caution, since the bone marrow is an immune-privileged organ with MSCs as the principal cellular immunomodulators. MSCs act to prevent an exacerbated inflammatory milieu within the bone marrow, since such a microenvironment could have detrimental effects on HSCs, immune cell development and viral clearance.

AMD3100 can mobilize hematopoietic stem cells (HSCs). This poses some concerns if this agent is used in patients to target bone marrow-resident breast cancer cells. If AMD3100 were to be used in combination with chemotherapy, then one potential side effect would be mobilization of HSCs. Additional antagonists for CXCR4 should be studied for those that can facilitate targeting of breast cancer cells but minimize toxicity to resident HSCs.

The findings on CXCL12/CXCR4 axis suggest that this is a druggable target to combat breast cancer metastasis to bone marrow, and other organs. It is possible that CXCR4 antagonists can induce re-cycling of breast cancer cells that interact with MSCs within the bone marrow, as well as within the tumor (FIG. 7), which is particularly relevant to the bone marrow, where MSCs reside around blood vessels and the trabeculae. Such an approaches can lead to safe and effective therapies by specifically targeting the mechanisms of breast cancer cells quiescence within the bone marrow, without deleteriously affecting HSCs or MSCs, and indirectly protecting the bone marrow microvasculature.

Clinical evidence indicates that breast cancer cells can remain quiescent without clinical evidence, and in bone marrow, without affecting hematopoiesis. The dormant breast cancer cells could resurge more than ten years later, with the re-animated breast cancer cells metastasizing to tertiary sites. Breast cancer cells can interact with (MSCs) through membrane-bound CXCL12 (SDF-1α) and its receptor, CXCR4. MSCs can also protect breast cancer cells from immune clearance through suppression of NK and CTL activity, as well as expansion of T-regulator cells (Tregs). However, since MSCs can also support the growth of breast cancer cells, the present invention is based upon the novel hypothesis that MSC-derived cytokine production could explain these differences. In the presence of MSCs, the CXCR4 antagonist, AMD3100, transitioned breast cancer cells into cycling cells and conferred susceptibility to carboplatin. The proliferation of breast cancer cells depended on the release of IL-1α and IL-1β from MSCs, but only if AMD3100 uncoupled breast cancer cells from MSCs. The findings which led to the present invention were validated in nude BALB/c where AMD3100 and carboplatin showed significant reduction in tumor volume, despite the presence of MSCs. However, when the activity of IL-1 was blocked with a receptor antagonist, the cancer cells resisted carboplatin. This verified a critical role for IL-1 for MSCs to transition from protective to supporting the growth of breast cancer cells. These findings are essential to the present invention, a novel, efficient immune therapy, in light of MSCs having a central role not only in breast cancer, but also in other solid tumors.

As part of the present invention, the CXCL12/CXCR4 interaction in breast cancer cell quiescence was elucidated. Breast cancer cells and MSCs interact through membrane-bound CXCL12 and CXCR4. The proliferation of breast cancer cells, in the presence or absence of MSCs, was compared and then the extent of involvement by the CXCL12/CXCR4 interaction was determined. The breast cancer cells and MSCs were studied as untransfected and CXCL12 or CXCR4 knockout (FIGS. 1A and 1B). Untransfected cells were co-cultured for 48 hours (FIG. 1C, second group from left) and the results showed significantly (p<0.05) reduced proliferation as compared to breast cancer cells cultured alone (FIG. 1C, far left bars). The reduction in breast cancer cell proliferation in the untransfected co-cultures cannot be explained by cell death, since there was no significant (p>0.05) difference in cell viability (not shown).

Similar studies were also conducted with MSCs and breast cancer cells, stably knockdown for CXCL12 and CXCR4. The knockdown of CXCL12 and/or CXCR4 in both breast cancer cells and MSCs (FIG. 1B: far right set of bars) produced a significant (p<0.05) increase in proliferation in both MDA-MB-231 (open bar) and T47D (diagonal bar). The reduction in proliferation was not due to change in cell viability (results not shown). These results indicate that the CXCL12/CXCR4 interaction between breast cancer cells and MSCs is important for maximal proliferation of breast cancer cells.

Pharmacological disruption of CXCL12/CXCR4 in breast cancer cell proliferation was necessarily involved with the conception of the present invention. The study of the molecular disruption of CXCL12-CXCR4 with a CXCR4 antagonist, AMD3100 was expanded. We first examined the effects of AMD3100 on the proliferation of MDA-MB-231 and T47D, in co-culture with MSCs (FIGS. 1D and 1E), in the presence of two different concentrations of the antagonist (10 and 100 ng/ml) for the 48-hour culture. Parallel cultures contained vehicle. The antagonist promoted the proliferation of breast cancer cells in co-cultures with MSCs (FIGS. 1D and 1E, closed circles), as compared to breast cancer cells alone (open circles). T47D showed less sensitivity (100 ng/ml) to AMD3100 as compared to MDA-MB-231 (10 ng/ml).

Breast cancer cells alone showed a decrease in cell proliferation with the antagonist (open circles). In contrast to MDA-MD-231, T47D proliferation was inversely related to AMD3100 concentration (FIGS. 1D and 1E, closed circles). The effects cannot be explained by changes in viability. Repeat studies at earlier time points verified 48 hours as optimum for AMD3100-mediated proliferation (FIGS. 1F and 1G, open circles). The increase in proliferation, however, began at 1 hour for MDA-MB-231, but at a later time point for T47D. There was no change in cell viability. These results indicate that pharmacological intervention with a CXCR4 antagonist is able to disrupt MSC-mediated breast cancer cell growth arrest at doses tolerable by the cells.

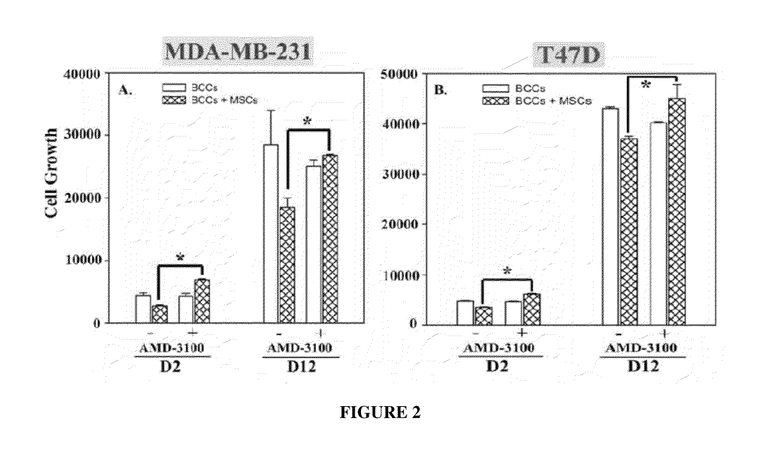

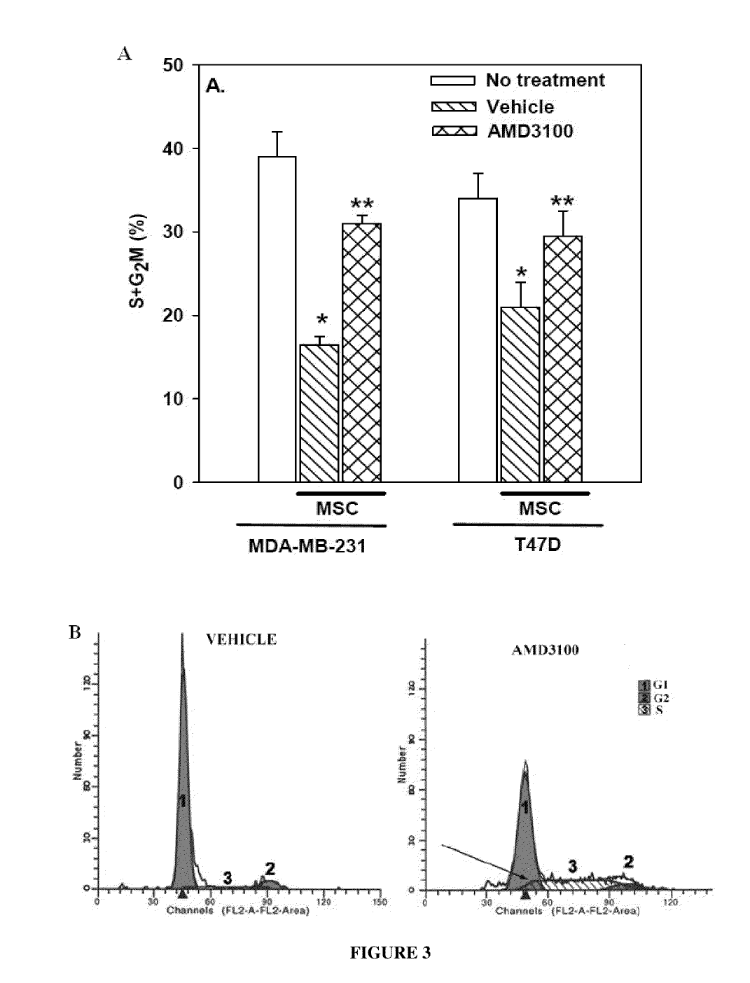

Some studies also looked at AMD3100 in long-term breast cancer cell/MSC co-cultures. In this set of studies we determined the effect of AMD3100 when breast cancer cells were cultured with MSCs for over 48 hours since this will recapitulate in situ when breast cancer cells are expected to contact MSCs for long periods. Breast cancer cells were co-cultured with MSCs for 12 days (D12), with optimal AMD3100 (FIG. 1) added during the last 48 hours. Control cultures contained vehicle (−) and 2-day exposure to AMD3100 (D2). For both cell lines, D12 co-cultures showed significant (p<0.05) decrease in proliferation as compared to breast cancer cells cultured alone (FIG. 2). This decrease was significantly (p<0.05) reversed with AMD3100 (FIG. 2). The changes in proliferation could not be explained by differences in cell viability (FIG. 3S). In total, the results, combined with those in FIG. 1 indicate that disruption of MSC-mediated breast cancer cell growth arrest in short- and long-term co-cultures could be clinically relevant to transition breast cancer cells into cycling cells for targeting with anti-cancer agent.

It was also necessary to understand the role of AMD3100 in breast cancer cell cell cycle re-entry. We studied cell cycle phase of 12-day co-cultured breast cancer cells, with or without AMD3100, as described for FIG. 2. Breast cancer cells cultured alone served as control. Propidium iodide DNA analyses with breast cancer cells, in co-culture with MSCs resulted in significantly (p<0.05) reduced cycling cells (S/G2/M phase) as compared to breast cancer cells alone (FIG. 3A). Treatment with AMD3100 produced a significant (p<0.05) increase in cycling cells compared to no treatment. FIG. 3B illustrates a representative histogram of the proportion of cycling (peaks 2 and 3) versus non-cycling (peak 1) MDA-MB-231 co-cultured with MSCs, with (right) or without AMD3100 (left). These data indicate that AMD3100 is able to disrupt MSC-mediated breast cancer cell growth arrest by promoting cell cycle re-entry.

Soluble factors in re-entry of breast cancer cell into cycling were also examined. AMD3100 promotes proliferation (FIG. 2) and cell cycle re-entry (FIG. 3) of breast cancer cells, in co-culture with MSCs. Using mechanistic studies, we next asked whether breast cancer cells proliferation by AMD3100 requires contact between MSCs and breast cancer cells. A contact-independent mechanism would suggest the involvement of soluble factors released by one or both cell types to induce proliferation. To address this question, we utilized a transwell assay in which “naïve” breast cancer cells, which were not previously exposed to MSCs or AMD3100, were seeded in the outer wells. MSCs from co-cultures with AMD3100 or vehicle were added to the inner wells. In parallel, only vehicle was added to the inner wells. There was a significant (p<0.05) increase in “naïve” breast cancer cell proliferation in transwells with MSCs from co-cultures with AMD3100 (FIGS. 4A and 4B, middle hatched bars) as compared to antagonist-treated MSCs alone (left hatched bars).

IL-1α in AMD3100-mediated breast cancer cell cycling is very important to the present invention. To assess the factor(s) responsible for breast cancer cell proliferation (FIGS. 4A and 4B), media from representative (MDA-MB-231) transwell cultures were analyzed with a cytokine/growth factor microarray. The pro-inflammatory cytokines IL-1α and IL-1β (FIG. 4C) were 3- and 5-fold greater, respectively, when MSCs were obtained from AMD3100-treated co-cultures. The involvement of IL-1α and IL-1β in the proliferation of breast cancer cells in the outer wells (FIGS. 4A and 4B) was determined by repeating the assay with IL-1RI neutralizing antibody (FIG. 4D). IL-1R1 significantly (p<0.05) impeded the proliferation of breast cancer cells (middle group, far right bar) as compared to non-immune IgG (middle group, second bar from left). IL-1RI had no effect on proliferation of “naïve” breast cancer cells incubated with MSCs previously cultured alone (left group of bars).

In pursuit of the present invention, we next identified whether MSCs and/or breast cancer cells are the sources of IL-1α and IL-1β, by repeating the transwell assay. Instead, we added “naïve” breast cancer cells, knockdown for IL-1α and IL-1β, or wild-type (FIG. 4E). The rationale for this approach is that if breast cancer cells rather than MSCs are the source of IL-1, its knockdown should have a similar effect on proliferation as the neutralizing antibody. Addition of AMD3100-treated MSCs from co-cultures (middle group of bars) to the inner wells produced no discernible difference in proliferation of wild-type and knockdown breast cancer cells, indicating that MSCs, and not breast cancer cells are the source of IL-1. In summary, the data indicate that treatment of MSC/breast cancer cell co-cultures with AMD3100 causes the release of IL-1α and IL-1β from MSCs, which in turn induces breast cancer cell cycle re-entry and proliferation.

As part of developing novel treatment approaches, AMD3100-mediated susceptibility of breast cancer cells to carboplatin was integral. The final set of studies determined the clinical relevance of AMD3100 as a breast cancer therapeutic by examining its ability to promote susceptibility to the chemotherapeutic, carboplatin, despite the presence of MSCs. Breast cancer cells co-cultured with MSCs (FIGS. 5A-5D; hatched bars) were chemoresistant (middle sets of bars) to carboplatin as compared to breast cancer cells cultured alone (open bars). This resistance was compromised when chemotherapy was added with AMD3100 (right sets of bars). Decreased viability was also confirmed by increased caspase-3 immunoreactivity (FIG. 5E) in breast cancer cells isolated from co-culture, hence demonstrating induction of apoptosis. Interestingly, when co-cultures were pre-treated with AMD3100 for 48 hours and then given chemotherapy in the absence of the antagonist, the effect on cell proliferation and viability was lost (FIG. 6; right sets of hatched bars). These findings indicate that AMD3100 needs to be included within the chemotherapeutic cocktail to be effective at inducing cell death in quiescent bone marrow breast cancer cells. In this light, AMD3100 may serve as an adjuvant treatment to chemotherapy in treating breast cancer.

### Carboplatin and AMD3100 Treatment in Tumor Volume

To validate our in vitro studies, female nude BALB/c mice were injected subreast cancerutaneously with 10⁶ matrigel-resuspended MDA-MB-231 alone or in combination with 10⁶ MSCs, as per the experimental design described in FIG. 7A. Mice were divided into the following experimental groups (n=5 per group): (A) breast cancer cells injected alone; (B) breast cancer cells injected in combination with MSCs; (C) group B plus AMD3100 treatment; (D) group C plus IL-1ra co-treatment. All groups were treated with carboplatin at days 8 and 10 following initial injection of cells, and the animals euthanized at the experimental endpoint of day 12 (D12). Mice injected with breast cancer cells alone (FIG. 7B, far left group of bars) showed a drastic reduction in mean tumor volume (solid bar) by D12, which was significantly (p<0.05) impeded by co-injection with MSCs (second group of bars from left). Pre-conditioning the tumors with AMD3100 significantly (p<0.05) reversed the chemoprotective effect of MSCs (second group of bars from right), although co-treatment with IL-1ra negated this response (far right group of bars). These results using an in vivo tumor model validate our in vitro studies (FIGS. 4 and 5), and suggest that AMD3100 disables the chemoprotective effect of MSC-breast cancer cells interaction through an IL-1-dependent mechanism.

The chemotherapeutic agents and the CXCR4 antagonists can be administered by means known in the art, such as intravenously or by infusion in pharmaceutically acceptable carriers as are known in the art.

### Materials and Methods

### Reagents and Antibodies

All tissue culture media were purchased from Gibreast cancero (Grand Island, N.Y.), fetal calf serum (FCS) from Hyclone Laboratories (Logan, Utah), IL-1α/β siRNA, Ficoll-Hypaque and AMD3100 from Sigma (St. Louis, Mo.), propidium iodide from BD Biosciences (San Jose, Calif.) and carboplatin from Teva Parenteral Medicines (Irvine, Calif.).

Goat anti-SDF-1α and anti-IL-1RI from R&D Systems (Minneapolis, Minn.), rabbit anti-CXCR4 from Affinity Bioreagents (Golden, Colo.), rabbit anti-caspase-3 from BD Pharmingen (San Diego, Calif.), mouse anti-cytokeratin, -β-actin mAb, horseradish-peroxidase (HRP)-conjugated anti-rabbit, -goat and -mouse IgG were purchased from Sigma. CellTiter-Blue cell viability assay was purchased from Promega (Madison, Wis.) and CyQUANT Cell Proliferation kit from Invitrogen (Carlsbad, Calif.).

### Cell Lines

T47D and MDA-MB-231 were purchased from American Type Culture Collection (ATCC; Manassas, Va.) and cultured in accordance with manufacturer's instructions.

### Culture of Human MSCs

MSCs were cultured from bone marrow aspirates. The use of human bone marrow aspirates followed a protocol approved by the Institutional Review Board of The University of Medicine and Dentistry of New Jersey-Newark Campus. Unfractionated bone marrow aspirates were cultured in DMEM with 10% FCS (D10 media) in Falcon 3003 dishes. After 3 days, red blood cells and granulocytes were removed with Ficoll Hypaque. After four cell passages, the adherent cells were asymmetric, CD14−, CD29+, CD44+, CD34−, CD45−, SH2+, prolyl-4-hydroxylase.

### Stable SDF-1α/CXCR-4 Knockdown

The shRNA vector, pPMSKH1-SDF-1/KC (wild-type and mutant), for CXCL12 was previously described in the art. pSUPER-CXCR4 (wild-type and mutant) shRNA vector for knockdown of CXCR4 was kindly provided by Dr. Si-Yi Chen (Baylor University). Breast cancer cells or MSCs were co-transfected with pTK-Hyg and pPMSKH1-SDF-1/KC or pSUPER-CXCR4 (both either wild-type or mutant) and then selected with hygromycin or G418. All knockdown cultures were maintained in media containing hygromycin. Levels of CXCL12 and CXCR4 protein expression were determined by western blot to validate knockdown.

### Western Analysis

Whole cell extracts from breast cancer cells and MSCs were prepared in accordance with accepted methods and 20 μg were analyzed by western blots using 4-20% SDS-PAGE (Bio-Rad, Hercules, Calif.). The proteins were transferred onto polyvinylidene difluoride membranes (Perkin Elmer Life Sciences, Boston, Mass.). Membranes were incubated overnight with primary antibodies and then detected the following day by 2 hour incubation with HRP-conjugated IgG. All primary and secondary antibodies were used at final dilutions of 1/1000 and 1/2000, respectively. HRP was developed with chemiluminscence detection reagent (Perkin Elmer Life Sciences). The membranes were stripped with Restore Stripping Buffer (Pierce, Rockford, Ill.) for reprobing with other antibodies.

### Proliferation/Viability

Cultures of breast cancer cells and MSCs, alone or in co-culture, from each experimental setup were assayed for cellular proliferation and viability using the CyQuant Cell Proliferation Assay Kit (Molecular Probes; Eugene, Oreg.) and CellTiter-Blue Cell Viability Assay (Promega), respectively, according to manufacturer's specific instructions.

For determination of proliferation, cells grown in 96-well plates were frozen overnight at −70° C. The next day, thawed cells were incubated in CyQuant GR dye/cell-lysis buffer for 5 minutes at room temperature, and examined using a fluorescence microplate reader at 480 nm excitation/520 nm emission. Proliferation was calculated from a standard curve of known numbers of breast cancer cells and MSCs. To accurately assess the proliferation of breast cancer cells, but not MSC proliferation in co-culture, proliferation of MSCs grown alone were subtracted from the total cellular proliferation recorded.

For assessment of viability, CellTiter-Blue reagent was added to cells grown in 96-well plates, and then incubated for 4 hours at 37° C. Following incubation, wells were read using a fluorescence microplate reader at 560 nm excitation/590 nm emission. Percent viability was calculated from a reference using untreated healthy cells, which were considered 100% viable, and cell-free wells containing reagent alone, which were considered 0% viable.

### Cell Cycle Analyses

Breast cancer cells and MSC co-cultures were incubated with anti-cytokeratin primary and FITC-anti IgG secondary antibodies to label the breast cancer cell fraction. Cells were then treated with RNase A (1 mg/ml) and fixed with cold 70% ethanol. Cells were stained with 20 μg/ml propidium iodide (PI) solution and transferred to round bottom tubes for DNA analysis by BD FACScan. Double positive cells identified only the desired cellular fraction, consisting of breast cancer cells that incorporated the DNA dye. Cultures incubated with FITC anti-IgG alone were used as isotype controls. All analyses were performed using BD CellQuest software and percent statistics were given.

### Transwell Assay

Breast cancer cells (5×10⁴) were added to the outer well of 24-well transwell cultures, 0.4 μM insert (BD Falcon). In parallel, breast cancer cells were separated from co-cultures with MSCs by positive selection with anti-cytokeratin-conjugated; pan anti-mouse IgG Dynabeads (Invitrogen). MSCs from the negative fraction were then added to the inner wells of the transwell chamber in order to determine the effects of MSC-derived soluble factors on breast cancer cell proliferation. For neutralization of soluble IL-1α and IL-1β, anti-IL-1RI was titrated into the 24-well plates. Neutralization of breast cancer cell proliferation was observed at a concentration of 1 μg/μl.

### Cytokine Array

Cytokine production by MSCs in the transwell assay was assessed using the Human Cytokine Antibody Array 5 (RayBiotech; Norcross, Ga.). Briefly, after 48 hours of culture, the transwell was removed and the breast cancer cell growth media collected for cytokine determination. Background levels obtained with media alone were subtracted. The densities of spots were quantitated with UN-SCAN-IT densitometry software (Silk Scientific; Orem, Utah). Cytokines demonstrating differential expression were normalized to internal positive controls and presented as fold change relative to an internal control, arbitrarily assigned a value of 1.

Transient Transfection of IL-1α/β siRNA

IL-1α and IL-1β siRNA duplexes (Sigma) were used to knockdown IL-1 production in breast cancer cells, prior to culture with MSC transwell inserts. MDA-MB-231 (5×10⁴) were seeded in 24-well plates, and after 24 hours, 100 nM IL-1α and IL-1β siRNA was delivered via DharmaFECT Transfection reagent (Dharmacon; Lafayette, Colo.). siRNA sequences were as follows: IL-1α|5′-guc auc aaa gga uga ugc u-3′|; IL-1β|5′-gau guc ugg ucc aua uga a-3′|. Knockdown was confirmed by PCR.

### Animal Studies

Female nude BALB/c mice (4 weeks) were obtained from Harlan Laboratories (Indianapolis, Ind.) and housed in a laminar flow hood at an AALAC-accredited facility. The use of mice was approved by the Institutional Animal Care and Use Committee, New Jersey Medical School (Newark, N.J.). MDA-MB-231 (10⁶), alone or in combination with MSCs (10⁶), in 0.1 mL and equal volume of BD Matrigel (BD Biosciences, Bedford, Mass.) were injected by subreast cancerutaneous route in the nude mice (Day 0, D0). After 4 days, the tumors were injected with AMD3100 alone or in combination with IL-1ra or vehicle (hereafter termed ‘treatment’). Mice were given a second ‘treatment’ at D8 along with a first dose of chemotherapy delivered intra-peritoneally. A second dose of chemotherapy was given at D10 and the mice euthanized at D12.

### Statistical Analysis

Statistical data analyses were performed with analysis of variance and Tukey-Kramer multiple comparisons test. p<0.05 was considered significant.

### Results

### CXCL12/CXCR4 Interaction in Breast Cancer Cell Quiescence

FIG. 1 demonstrates the CXCL12-CXCR4 interaction between breast cancer cells and MSCs in breast cancer cell proliferation. Western blots were performed with extracts from MSCs and breast cancer cells, stably knockdown (KD) for CXCL12 (A) and CXCR4 (B). Controls included mutant (mut) shRNA and non-transfectants (NT). Membranes were stripped and re-probed with β-actin for normalization. MSCs and breast cancer cells, knockdown for CXCL12 or CXCR4 and controls were cultured alone or together for 48 hours and then studied for proliferation (C). The results are presented as mean±SD, n=3. Dose-response curves were established for AMD3100 on breast cancer cell proliferation, in the presence or absence of MSCs in 48 hour cultures (D and E). The results are presented as mean±SD, n=3. Optimal AMD3100 (D and E) dose points were selected and then studied in proliferation responses at various times (F and G) and the results are presented as mean±SD, n=3. (Note that the p value is less than or equal to 0.05 as opposed to breast cancer cells alone.)

### AMD3100 in Long-Term Breast Cancer Cell/MSC Co-Cultures

FIG. 2 illustrates the effect of AMD3100on breast cancer cell proliferation following extended co-culture with MSCs. MDA-MB-231 (A) or T47D (B), alone or with MSCs, were treated with AMD3100 for either 2 (D2) or 12 (D12) days. Control cultures were untreated. The results are presented as the mean±SD, n=3. The p value was less than or equal to 0.05, compared to breast cancer cells alone.

### The Role of AMD3100 in Breast Cancer Cell Cell Cycle Re-Entry

The effect on cycling of breast cancer cells by AMD3100 is shown in FIG. 3. Breast cancer cells, alone or 12-day cultures with MSCs were treated with AMD3100 for the last 48 hours or untreated. At the end of the assay, cells were co-labeled with anti-cytokeratin and propidium iodide and then analyzed by flow cytometry. Results are presented as the mean percent cycling cells (S+G2/M phases)±SD, n=3 (B). Also shown is a representative histogram for MDA-MB-231 co-cultured with MSCs, with or without AMD3100. The areas of histograms represent (1) non-cycling and (2, 3) cycling cells. Arrow shows the increase in cycling cells following AMD3100 treatment. Here, the p value is less than or equal to 0.05, as compared to breast cancer cells alone; the p value is less than or equal to 0.05 in comparison to breast cancer cells with MSCs; no AMD3100.

### IL-1α in AMD3100-Mediated Breast Cancer Cell Cycling

FIG. 4 shows IL-1 in AMD3100-mediated breast cancer cell cycling. MSCs were co-cultured with breast cancer cells for 12 days with, or without AMD3100. The MSCs were selected and then added to the inner wells of transwell cultures. Naïve breast cancer cells were placed in the outer wells. After 48 hours, breast cancer cells were counted and the results presented as mean±SD, n=3 (A and B). The media were analyzed for cytokine production with protein array and the normalized values presented as fold change relative to the internal control (C). The studies were repeated with anti-IL-1RI or control IgG (D); or with IL-1α/β knockdown MSCs (E). The results are presented as mean cell number±SD, n=3. P values are as follows: p≦0.05 vs. no AMD3100; **p≦0.05 vs. control IgG.

### AMD3100-Mediated Susceptibility of Breast Cancer Cells to Carboplatin

In FIG. 5, results are shown of the treatment of co-cultured breast cancer cells/MSCs with carboplatin and AMD3100. Breast cancer cells, cultured alone or with MSCs for 12 days, were treated with AMD3100, or untreated. After 48 hours, all cultures were exposed to carboplatin, with re-added AMD3100 in cultures that had initial AMD3100. After 72 hours, the viable cells were counted (A and B), and presented (C and D) as the mean±SD, n=3. Whole cell extracts from these experiments were analyzed by immunoblot for caspase-3 (E). Membranes were stripped and re-probed with β-actin for normalization. Representative blots are shown, n=3. Here, the p value was ≦0.05, compared to breast cancer cells alone and ≦0.05 compared to breast cancer cells and MSCs.

### Non-Combinatorial, AMD3100-Treated MSC/Breast Cancer Cell Co-Cultures

Similarly, in FIG. 6, results show the resistance to chemotherapy in non-combinatorial, AMD3100-treated MSC/breast cancer cell co-cultures. Breast cancer cells cultured in the presence or absence of MSCs for 12 days were treated for 48 hours with AMD3100, or untreated. Following 48 hours, treated and untreated cells were exposed to carboplatin for 72 hours to induce cell death. The treated cells did not receive chemotherapy in combination with a second dose of AMD3100 (as illustrated by +/−). At the end of this regiment cell proliferation (A and B) and viability (C and D) were assessed. The results are presented as the mean±SD, n=3.

### Carboplatin and AMD3100 Treatment in Tumor Volume

In FIG. 7, the effects of AMD3100 and carboplatin on tumor volume are represented. The method by which mice were treated is shown in (A). (B) Female BALB/c (n=5) were injected subreast cancerutaneously with matrigel and 10⁶ MDA-MB-231 alone or in combination with 10⁶ MSCs. The mean volumes at the first injection (day 8, D8) are normalized to 100% and the change in volumes at D10 and D12 are presented as mean±SD. (C) Schematic depicting mechanism of AMD3100 action in mediating breast cancer cells chemotherapeutic susceptibility following interaction with MSCs. breast cancer cells that have metastasized from the primary tumor site into the systemic circulation enter the central sinus of the bone marrow where they extravasate across the endothelium and come into contact with resident MSCs. Interaction between CXCL12, secreted by MSCs and/or breast cancer cells, and CXCR4, expressed on both cell types, facilitates breast cancer cells integration and dormancy within the bone marrow (1). Treatment with AMD3100 (2) breaks this chemokine ligand-receptor interaction and triggers the MSCs to release the inflammatory cytokines, IL-1α and IL-1β. Having been released from the MSCs, the breast cancer cells begin to cycle and proliferate, thus conferring susceptibility to chemotherapeutic treatment.

### REFERENCES

1. Braun, S., and B. Naume. 2005. Circulating and Disseminated Tumor Cells. J. Clin. Oncol. 23: 1623-1626.

2. Gluck, S. 1995. Autologous transplantation for patients with advanced breast cancer with emphasis on bony metastasis. Can. J. Oncol. 5 Suppl 1: 58-62.

3. Habeck, M. 2000. Bone-marrow analysis predicts breast-cancer recurrence. Mol. Med. Today 6: 256-257.

4. Mansi, J. L., U. Berger, T. McDonnell, A. Pople, Z. Rayter, J. C. Gazet, and R. C. Coombes. 1989. The fate of bone marrow micrometastases in patients with primary breast cancer. J. Clin. Oncol. 7: 445-449.

5. Patel, S. A., A. C. Heinrich, B. Y. Reddy, B. Srinivas, N. Heidaran, and P. Rameshwar. 2008. Breast cancer biology: the multifaceted roles of mesenchymal stem cells. J. Oncol. 2008: 425895.

6. Naume, B., X. Zhao, M. Synnestvedt, E. Borgen, H. G. Russnes, O. C. Lingjaerde, M. Stromberg, G. Wiedswang, G. Kvalheim, R. Karesen, J. M. Nesland, A. L. Borresen-Dale, and T. Sorlie. 2007. Presence of bone marrow micrometastasis is associated with different recurrence risk within molecular subtypes of breast cancer. Mol. Oncol. 1: 160-171.

7. Katz, D., and D. Aharoni. 2004. Lytic Lesions in Breast Cancer. N Engl J Med 351: 2850.

8. Dai, H., L. van't Veer, J. Lamb, Y. D. He, M. Mao, B. M. Fine, R. Bernards, M. van de Vijver, P. Deutsch, A. Sachs, R. Stoughton, and S. Friend. 2005. A Cell Proliferation Signature Is a Marker of Extremely Poor Outcome in a Subpopulation of Breast Cancer Patients. Cancer Res. 65: 4059-4066.

9. Corcoran, K. E., N. Patel, and P. Rameshwar. 2007. Stromal Derived Growth Factor-1{alpha}: Another Mediator in Neural-Emerging Immune System through Tac1 Expression in Bone Marrow Stromal Cells. J. Immunol. 178: 2075-2082.

10. Moharita, A. L., M. Taborga, K. E. Corcoran, M. Bryan, P. S. Patel, and P. Rameshwar. 2006. SDF-1{alpha} regulation in breast cancer cells contacting bone marrow stroma is critical for normal hematopoiesis. Blood 108: 3245-3252.

11. Oh, H. S., A. Moharita, J. G. Potian, I. P. Whitehead, J. C. Livingston, T. A. Castro, P. S. Patel, and P. Rameshwar. 2004. Bone Marrow Stroma Influences Transforming Growth Factor-{beta} Production in Breast Cancer Cells to Regulate c-myc Activation of the Preprotachykinin-I Gene in Breast Cancer Cells. Cancer Res. 64: 6327-6336.

12. Corcoran, K. E., K. A. Trzaska, H. Fernandes, M. Bryan, M. Taborga, V. Srinivas, K. Packman, P. S. Patel, and P. Rameshwar. 2008. Mesenchymal Stem Cells in Early Entry of Breast Cancer into Bone Marrow. PLoS ONE 3: e2563.

13. Patel, S. A., J. R. Meyer, S. J. Greco, K. E. Corcoran, M. Bryan, and P. Rameshwar. 2010. Mesenchymal Stem Cells Protect Breast Cancer Cells through Regulatory T Cells: Role of Mesenchymal Stem Cell-Derived TGF-{beta}. J. Immunol. 184: 5885-5894.

14. Dennis, J. E., and P. Charbord. 2002. Origin and differentiation of human and murine stroma. Stem Cells 20: 205-214.

15. Helbig, G., W. Kent, P. Bhat-Nakshatri, S. Kumar, H. Kishimoto, K. D. Miller, H. E. Broxmeyer, and H. Nakshatri. 2003. NF-kB Promotes Breast Cancer Cell Migration and

Metastasis by Inducing the Expression of the Chemokine Receptor CXCR4. J. Biol. Chem. 278: 21631-21638.

16. Muller-Sieburg, C. E., and E. Deryugina. 1995. The stromal cells' guide to the stem cell universe. Stem Cells 13: 477-486.

17. Orimo, A., P. B. Gupta, D. C. Sgroi, F. Arenzana-Seisdedos, T. Delaunay, R. Naeem, V. J. Carey, A. L. Richardson, and R. A. Weinberg. 2005. Stromal fibroblasts present in invasive human breast carcinomas promote tumor growth and angiogenesis through elevated SDF-1/CXCL12 secretion. Cell 121: 335-348.

18. Rao, G., P. S. Patel, S. P. Idler, P. Maloof, P. Gascon, J. A. Potian, and P. Rameshwar. 2004. Facilitating Role of Preprotachykinin-I Gene in the Integration of Breast Cancer Cells within the Stromal Compartment of the Bone Marrow: A Model of Early Cancer Progression. Cancer Res. 64: 2874-2881.

19. Deans, R. J., and A. B. Moseley. 2000. Mesenchymal stem cells: biology and potential clinical uses. Exp. Hematol. 28: 875-884.

20. von Tell, D., A. Armulik, and C. Betsholtz. 2006. Pericytes and vascular stability. Exp. Cell Res. 312: 623-629.

21. Potian, J. A., H. Aviv, N. M. Ponzio, J. S. Harrison, and P. Rameshwar. 2003. Veto-Like Activity of Mesenchymal Stem Cells: Functional Discrimination Between Cellular Responses to Alloantigens and Recall Antigens. J. Immunol. 171: 3426-3434.

22. Foss, B., T. Hervig, and O. Bruserud. 2009. Connexins are active participants of hematopoietic stem cell regulation. Stem Cells Dev. 18: 807-812.

23. Greco, S. J., C. Zhou, J. H. Ye, and P. Rameshwar. 2007. An Interdisciplinary Approach and Characterization of Neuronal Cells Transdifferentiated from Human Mesenchymal Stem Cells. Stem Cells and Dev. 16: 811-826.

24. Lapteva, N., A. G. Yang, D. E. Sanders, R. W. Strube, and S. Y. Chen. 2004. CXCR4 knockdown by small interfering RNA abrogates breast tumor growth in vivo. Cancer Gene Ther. 12: 84-89.

25. 2007. Plerixafor: AMD 3100, AMD3100, JM 3100, SDZ SID 791. Drugs R. D. 8: 113-119.

26. Mirisola, V., A. Zuccarino, B. E. Bachmeier, M. P. Sormani, J. Falter, A. Nerlich, and U. Pfeffer. 2009. CXCL12/SDF1 expression by breast cancers is an independent prognostic marker of disease-free and overall survival. Eur. J. Cancer 45: 2579-2587.

27. Smith, M. C. P., K. E. Luker, J. R. Garbow, J. L. Prior, E. Jackson, D. Piwnica-Worms, and G. D. Luker. 2004. CXCR4 Regulates Growth of Both Primary and Metastatic Breast Cancer. Cancer Res. 64: 8604-8612.

28. Teicher, B. A., and S. P. Fricker. 2010. CXCL12 (SDF-1)/CXCR4 Pathway in Cancer. Clin. Cancer Res. 16: 2927-2931.

29. Kalatskaya, I., Y. A. Berchiche, S. p. Gravel, B. J. Limberg, J. S. Rosenbaum, and N. Heveker. 2009. AMD3100 Is a CXCR7 Ligand with Allosteric Agonist Properties. Mol. Pharmacol. 75: 1240-1247.

30. Chan, J. L., K. C. Tang, A. P. Patel, L. M. Bonilla, N. Pierobon, N. M. Ponzio, and P. Rameshwar. 2006. Antigen-presenting property of mesenchymal stem cells occurs during a narrow window at low levels of interferon-{gamma}. Blood 107: 4817-4824.

31. Kang, H. S., M. Habib, J. Chan, C. Abavana, J. A. Potian, N. M. Ponzio, and P. Rameshwar. 2005. A paradoxical role for IFN-gamma in the immune properties of mesenchymal stem cells during viral challenge. Exp. Hematol. 33: 796-803.

32. Tang, K. C., K. A. Trzaska, S. V. Smirnov, S. V. Kotenko, S. K. Schwander, J. J. Ellner, and P. Rameshwar. 2008. Down-Regulation of MHC II in Mesenchymal Stem Cells at High IFN-{gamma} Can Be Partly Explained by Cytoplasmic Retention of CIITA. J Immunol. 180: 1826-1833.

33. Sakaguchi, Y., I. Sekiya, K. Yagishita, S. Ichinose, K. Shinomiya, and T. Muneta. 2004. Suspended cells from trabecular bone by collagenase digestion become virtually identical to mesenchymal stem cells obtained from marrow aspirates. Blood 104: 2728-2735.

We claim:

1. A method of treating breast cancer which comprises dissociating quiescent breast cancer cells from their environment in the bone marrow and treating the disassociated breast cancer cells with standard chemotherapeutic agents.

2. The method of claim 1 which comprises using a CXCR4 antagonist to break a BCC-MSC association, thus dissociating the quiescent breast cancer cells and inducing breast cancer cell proliferation and susceptibility to chemotherapy.

3. The method of claim 2 wherein the CXCR4 antagonist is AMD3100.

4. The method of claim 2 wherein the chemotherapeutic agent is carboplatin.

5. The method of claim 2 wherein the CXCR4 antagonist and the chemotherapeutic agent are administered simultaneously.
